(19) United States
(12) Patent Application Publication
(10) Pub. No.: US 2004/0029192 A1
(43) Pub. Date: Feb. 12, 2004

(54) Compositions and methods for the diagnosis and treatment of kidney disease

(76) Inventors: Andrey S. Shaw, St. Louis, MO (US); Jeffrey H. Miner, St. Louis, MO (US)

Correspondence Address: Joseph A. Mahoney, Mayer, Brown, Rowe, & Maw, P.O. Box 2828, Chicago, IL, 60690-2828, US

(21) Appl. No.: 10/215,126

(22) Filed: Aug. 8, 2002

(51) Int. Cl. G01N 33/567

(52) U.S. Cl. 435/7.21

(57) ABSTRACT

Polymorphisms associated with the CD2AP protein are disclosed. Compositions and methods for the diagnosis and treatment of kidney disease are also disclosed.

### GOVERNMENT INTEREST

This invention was partly supported by grants from the National Institutes of Health NIAID Grant No. ROIAI34094-06; therefore, the government has certain rights to the invention.

### FIELD OF THE INVENTION

The present invention relates to compositions and methods for diagnosing and treating kidney disease.

### BACKGROUND OF THE INVENTION AND RELATED ART

CD2-associated protein (CD2AP) is an 80-kilodalton protein that is critical for stabilizing contacts between T cells and antigen-presenting cells. It is an adapter protein that interacts with the cytoplasmic domain of CD2. CD2, a T cell and natural killer cell membrane protein, facilitates T cell adhesion to antigen-presenting cells. CD2AP enhances CD2 clustering and anchors CD2 at sites of cell contact. See Dustin et al., A Novel Adaptor Protein Orchestrates Receptor Patterning and Cytoskeletal Polarity in T-cell Contacts, CELL 94:667-77 (Sep. 4, 1998); Shih, Congenital Nephrotic Syndrome in Mice Lacking CD2-Associated Protein, SCIENCE 286:312-15 (Oct. 8, 1999).

The present invention is directed to isolated and purified CD2AP single nucleotide polymorphisms (SNPs) and other polymorphisms, and to the characterization and uses of such polymorphisms. Further, the present invention is directed to monoclonal antibodies that are specific for CD2AP in the diagnosis of kidney disease.

Glomerular diseases are extremely prevalent within the general population, and many cases are likely to have a genetic component. Inheritance of a single mutated allele could result in decreased protein expression of a critical podocyte protein. Normally, this would not result in any obvious defects. Insults to the kidney, however, such as drugs or infectious agents or the loss of glomeruli with aging, may place increased stress on the remaining glomeruli. The genetic mutation may then be revealed as an enhanced sensitivity to a renal insult. Therefore, there is a need to identify the genetic bases of glomerular diseases.

### SUMMARY OF THE INVENTION

Disclosed herein are methods for screening patients for a genetic susceptibility to kidney disease due to mutations in the CD2AP gene. The inventive methods are also useful for testing patients with kidney disease to determine whether the disease is due to a mutation in CD2AP.

The experiments disclosed herein support the role of CD2AP in podocyte function and glomerular disease. Polymorphisms associated with CD2AP are disclosed herein. One aspect of the present invention is directed to such polymorphisms isolated from a tissue specimen and the method of their identification. In an additional aspect, the present invention relates to antibodies (and to hybridomas which synthesize and secrete monoclonal antibodies) specific for CD2AP to measure its pattern of expression as an additional tool to screen for and/or diagnose kidney disease. Accordingly, inventive compositions comprising an antibody specific for CD2AP are also disclosed.

In a further aspect, the present invention is directed to various methods of utilizing the polymorphisms and the antibodies of the present invention for therapeutic and/or diagnostic purposes. For example, CD2AP polymorphisms and antibodies have use in: (1) diagnosing and treating focal and segmental glomerulosclerosis (“FSGS”); and (2) diagnosing and treating glomerular and other kidney diseases.

### BRIEF DESCRIPTION OF THE DRAWINGS

FIG. 1 is a drawing illustrating the polymorphisms identified in the CD2AP gene.

FIG. 2 is a series of photomicrographs showing the expression of CD2AP and nephrin in 2-and 7-week-old mouse glomeruli. Sections of 2- (A-D) and 7-week-old (E-H) kidneys from CD2AP ± and −/− mice were stained with CD2AP or nephrin antisera, as indicated. CD2AP is undetectable in the −/− kidneys (C and G). In the mutant, nephrin appears to be expressed normally at 2 week (D) but is only detected segmentally in a very few glomeruli at 7 week (H). Arrowheads in H point to podocytes lacking nephrin. Bar, 50 μ.m.

FIG. 3 is a series of photomicrographs showing the aberrant localization of CD2AP in Lamb2 mutant glomeruli. Sections of 3-week-old kidney from a control and a mutant lacking the laminin-β₂ chain were stained with the CD2AP antiserum. Much of the CD2AP immunoreactivity is punctate in the mutant (B and D) but not in the control (A and C). The mutant exhibits nephrotic syndrome and extensive foot process effacement at this age. Bar: 33 μ.m. for A and B; 16 μ.m. for C and D.

FIG. 4 is a series of photomicrographs showing the extraglomerular expression of CD2AP in mouse kidney. Sections of newborn (A-C) and 2-week-old (D-I) kidneys were labeled with the indicated antibodies or DBA lectin. A and B: in the nephrogenic zone, CD2AP is expressed in the Dolichos biflorus agglutinin (DBA)-positive ureteric bud, but staining is weak at the branching tip (t). C: medullary collecting ducts are strongly positive for CD2AP. D-I: in cortex, CD2AP is concentrated apically in cells of some proximal tubules (which are laminin-α₁ positive; arrowheads) and distal tubules (laminin-α₁ negative; arrows) but is mostly diffuse in collecting duct cells (*). Bar: 100 μ.m. for I; 50 μ.m. for all other panels.

FIG. 5 is a series of photomicrographs showing the immunoelectron microscopic localization of CD2AP in a 6-week-old normal mouse was performed using affinity purified CD2AP and secondary antibody conjugated with 10 nm-gold particles. The immunogold particles (arrow heads) were found predominantly along the lateral borders of the podocyte foot processes (B-D). Many of the particles localize in a region near or at the slit diaphragm. Original magnification, ×20,000. (A) is the control.

### DETAILED DESCRIPTION

As embodied and broadly described herein, the present invention is directed to methods for predicting a patient's predisposition toward developing kidney disease, to diagnosing kidney disease, to treating it, and to antibodies, diagnostic kits, oligonucleotide probes and other reagents that can be used with these methods.

A. Definitions

For convenience, the meaning of certain terms and phrases employed in the specification, examples, and appended claims are provided below:

“Allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

“Antibody” refers to polyclonal antibodies, monoclonal antibodies, entire immunoglobulin or any functional fragment. The term also encompasses fragments, like Fab and F(ab′)₂, of CD2AP antibodies, and conjugates of such fragments, and so-called “antigen binding proteins” (single-chain antibodies) which are based on CD2AP antibodies, in accordance, for example, with U.S. Pat. No. 4,704,692, incorporated herein by reference.

“CD2AP gene” means generically the CD2AP B gene, and its alternate forms include splicing variants and polymorphisms.

“CD2AP nucleic acid” refers to a nucleic acid encoding CD2AP, as well as fragments, homologs, complements, and derivatives thereof.

“CD2AP polypeptide” and “CD2AP protein” are intended to encompass polypeptides comprising the amino acid sequence, or fragments, homologs, complements, and derivatives thereof.

“CD2AP polymorphism or SNP” means one or more single nucleotide polymorphism for the CD2AP gene disclosed herein such as nucleic acids, as well as fragments, homologs, complements, and derivatives thereof.

“Encode” in its various grammatical forms as used herein includes nucleotides and/or amino acids that correspond to other nucleotides or amino acids in the transcriptional and/or translational sense, despite the fact that they may not strictly encode for one another.

“Gene chips” (also “gene arrays” and “lab on a chip”) means the covalent attachment of oligonucleotides or cDNA directly onto a small glass or silicon chip in organized arrays. These microdevices allow rapid, microanalytical analysis of DNA or protein in a single, fully integrated system. Typically, these devices are miniature surfaces, made of silicon, glass, or plastic, which carry the necessary microdevices (pumps, valves, microfluidic controllers, and detectors) that allow sample separation and analysis.

“Homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences.

“Isolated” as used herein with respect to nucleic acids, such as DNA or RNA, refers to molecules separated from other DNAs, or RNAs, respectively, which are present in the nature source of the macromolecule. Moreover, an “isolated nucleic acid” is meant to include nucleic acid fragments, which are not naturally occurring as fragments and would not be found in the nature state. The term “isolated” is also used herein to refer to polypeptides, which are isolated from other cellular proteins and is meant to encompass both purified and recombinant polypeptides.

“Kidney disease” as used herein refers to a host of ailments such as kidney disease, nephritic syndrome, glomerulonephritis (GN), end stage renal disease, membraneous glomerulonephritis, FSGS, minimal change disease, lupus glomerulonephritis, immunotactoid glomerulonephritis, and polycystic kidney disease (PKD).

“Marker” means regions of the DNA that vary between individuals. The different sequence variants at a given marker are called alleles or polymorphisms.

“Nucleic acid” refers to polynucleotides or oligonucleotides such as deoxyribonucleic acid (“DNA”), and, where appropriate, ribonucleic acid (“RNA”). The term should also be understood to include, as equivalents, analogs of either RNA or DNA made from nucleotide analogs and as applicable to the embodiment being described, single (sense or antisense) and double-stranded polynucleotides. The nucleic acids and amino acids, which occur in various amino acid sequences appearing herein, are identified according to their well-known, three letter or one letter abbreviations.

“Polymorphism” refers to the coexistence of more than one form of a gene or portion (e.g., allelic variant) thereof. A portion of a gene of which there are at least two different form, ie., two different nucleotide sequences, is referred to as a “polymorphic region of a gene.” A polymorphic region can be a single nucleotide, the identify of which differs in different alleles. A polymorphic region can be a single nucleotide, the identity of which differs in different alleles. A polymorphic region can also be several nucleotides long.

“Predisposition” or “Susceptibility” to disease means that certain alleles are discovered to be associated with a given disease state. They are thus over represented in individuals with disease as compared with health individuals. Therefore, the presence of such alleles indicates that an individual is at risk for the disease.

B. Anti-CD2AP: an Immunological Diagnostic for Kidney Disease Associated with Podocyte Damage

CD2AP plays a unique role in kidney function. As shown below (and detailed in Li et al., CD2AP Is Expressed With Nephrin In Developing Podocytes And Is Found Widely In Mature Kidney And Elsewhere, AM. J. PHYSIOL. RENAL PHYSIOL. 279:F785-F792 (Oct. 2000) and incorporated herein by reference), CD2AP is mislocalized in a mouse model of nephrotic syndrome (a type of kidney disease similar to a relatively common human disease, minimal change nephrotic syndrome). The ability to see mislocalization of CD2AP can be indicative of the kind of kidney malfunction occurring that cannot be determined without using expensive and time consuming electron microscopic methods. The present invention relates to an antibody to CD2AP for detecting CD2AP in glomerular podocytes, which are important in kidney function. As shown in the data below, when podocytes are damaged or not functioning properly, CD2AP is aberrantly localized in a punctate pattern. Therefore, anti-CD2AP may be utilized in a biopsy to rapidly determine whether podocytes are damaged, and precludes the necessity for performing other more costly diagnostic assays. Proteinuria is indicative of podocyte damages. While histological methods can be used to diagnose some glomerular diseases, some glomerular diseases like minimal change disease do not demonstrate any pathological change detectable by any current histological methods. Current methods require the use of electron microscopy to determine whether there is podocyte damage. Accordingly, the present invention is directed to antibodies to CD2AP and its use in a method for diagnosing glomerular dysfunction. The inventive method has further utility in assessing the amount of damage in a diseased kidney, and will allow clinicians to predict the outcome or likelihood of remission from nephrotic syndrome. Further, the method will help clinicians assess the effectiveness of a treatment by analyzing a post-treatment biopsy.

CD2AP mRNA is expressed widely in both mouse and human tissues (Dustin, et al., A Novel Adaptor Protein Orchestrates Receptor Patterning and Cytoskeletal Polarity in T-Cell Contacts, CELL 94:667-677 (1998); Kirsch, et al., CMS: An Adapter Molecule Involved in Cytroskeletal Rearrangements, PROC. NATL. ACAD. Sci. USA 96:6211-6216 (1999)). However, the most significant defect detected in mutant mice lacking CD2AP is in the kidney (Shih, et al., Congenital Nephrotic Syndrome in Mice Lacking CD2-Associated Protein, SCIENCE 286:312-315 (1999)). CD2AP -/- mice are a model for congenital nephrotic syndrome. They die at 6-7 weeks of age from proteinuria and renal failure. Histologically, there is effacement of podocyte foot processes beginning at 1 week of age, followed by excessive deposition of extracellular matrix by mesangial cells. This leads to a greatly expanded mesangium, distension of the glomerular capillary loops, and eventual blockage of the capillaries by matrix material. Immunohistochemical localization of CD2AP in normal kidney demonstrated that, in the glomerulus, it is restricted to podocytes in a pattern consistent with concentration in foot processes, suggesting that the mesangial expansion is secondary to a podocyte foot process defect.

Another molecule found in podocytes and associated with congenital nephrotic syndrome is nephrin. Nephrin, like CD2, is a transmembrane protein of the immunoglobulin superfamily. It is encoded by the gene NPHS1, which is mutated in congenital nephrotic syndrome of the Finnish type (Kestila, et al., Positionally Cloned Gene for a Novel Glomerular Protein-Nephrin-is Mutated in Congenital Nephrotic Syndrome, MOL. CELL 1:575-582 (1998); Tryggvason, et al., Discovery of the Congenital Nephrotic Syndrome Gene Discloses the Structure of the Mysterious Molecular Sieve of the Kidney, INT. J. DEV. BIOL. 43:445-451 (1999)). Nephrin has been localized to the glomerular slit diaphragm (Holthofer, et al., Nephrin Loclizes at the Podocyte Filtration Slit Area and is Characteristically Spliced in the Human Kidney, AM. J. PATHOL. 155:1681-1687 (1999); Holzman, et al., Nephrin Localizes to the Slit Porte of the Glomerular Epithelial Cell, KIDNEY INT. 56:1481-1491 (1999); Ruotsalainen, et al., Nephrin is Specifically Located at the Slit Diaphragm of Glomerular Podocytes, PROC. NATL. ACAD. SCI. USA 96:7962-7967 (1999)), suggesting that, together with the genetic data, nephrin is an essential component of this specialized structure. Analogous to its association with CD2, CD2AP can bind to the cytoplasmic tail of nephrin, suggesting that the foot process effacement in the CD2AP −/− kidney is related to the absence of CD2AP/nephrin interactions, which may be necessary to stabilize the slit diaphragm (Shih, et al., Congenital Nephrotic Syndrome in Mice Lacking CD2-Associated Protein, SCIENCE 286:312-315 (1999)).

To understand more about the biology of CD2AP and nephrin, Applicants determined and compared their patterns of expression in the developing mouse and human kidney. In addition, they examined CD2AP localization in a mouse model of nephrotic syndrome and have defined sites of CD2AP expression in other tissues.

Materials and Methods

1. Histology. CD2AP ± (control) and −/− tissues were obtained from mice that have been described previously (Shih, et al., Congenital Nephrotic Syndrome in Mice Lacking CD2-Associated Protein, SCIENCE 286:312-315 (1999)). Fetal human kidney at 83 days gestation was obtained from the University of Washington School of Medicine, Central Laboratory for Human Embryology (Seattle, Wash.). Tissues were frozen in OCT compound and sectioned at 7 μm on a cryostat. Sections were fixed in 2 or 4% paraformaldehyde for 10 minutes, rinsed in PBS, and blocked for 30 minutes in 1% goat serum, 1% BSA in PBS. Primary antibody diluted in 1% BSA in PBS was applied for 1 hour. Sections were rinsed in PBS, and then fluorophore-conjugated second antibody was applied for 1 hour. After rinsing in PBS, sections were mounted in 90% glycerol/0.1×PBS/1 mg/ml p-phenylenediamine. Sections were viewed under eipfluorescent illumination on a Nikon Eclipse 800 microscope. Images were captured with a Sport 2 cooled color digital camera (Diagnostic Instruments, Sterling Heights, Mich.) using Spot Software Version 2.1. Images were imported into Adobe Photoshop 5.0.2 for final processing and layout.

2. Antibodies and lectins. Rabbit anti-CD2AP antiserum has been previously described (Dustin, et al., A Novel Adaptor Protein Orchestrates Receptor Patterning and cytoskeletal Polarity in T-Cell Contacts, CELL 94:667-677, (1998)). Rabbit anti-nephrin antiserum was generated by immunization with a bacterial fusion protein containing portions of the human nephrin cytoplasmic tail (amino acids 1084-1241 in Krestila, et al., MOL. CELL 1:575-582 (1998)) and mouse dihydrofolate reductase (J. Patrakka, V. Ruotsalainen, P. Reponen, I. Ketola, C. Holmberg, M. Heikinheimo, K. Tryggvason, and H. Jalanko., unpublished observations). Mouse anti-synaptopodin monoclonal antibody (Mundel, et al., Podocytes in Glomerulus of Rat Kidney Express a Characteristic 44 KD Protein, J. HISTOCHEM. CYTOCHEM. 39:1047-1056 (1991)) was a gift from Peter Mundel (Albert Einstein College of Medicine, Bronx, N.Y.). Rat anti-mouse laminin-al, clone 8B3 (Abrahamson, et al., Selective Immunoreactivities of Kidney Basement Membranes to Monoclonal Antibodies Against Laminin: Localization of the End of the Long Arm and the Short Arms to Discrete Microdomains, J. CELL. BIOL. 109:3477-3491 (1989)), was a gift from Dale Abrahamson (Univ. of Kansas Medical Center, Kansas City, Kans.). Rat anti-mouse laminin-γ₁ (mAb-1914) and -β₁(mAb-1928) were from Chemicon (Temecula, Calif.). Rat anti-integrin-α₆, clone GoH3, and rat anti-platelet endothelial cell adhesion molecule were from Pharmingen (San Diego, Calif.). Rabbit anti-Tamm-Horsfall protein was from Biomedical Technologies (Stoughton, Mass.). FITC and Cy3-conjugated second antibodies were from ICN/Cappel (Costa Mesa, Calif.). FITC-conjugated Dolichos biflorus agglutinin and Lotus tetragonolobus lectin were from Vector Laboratories (Burlingame, Calif.).

Results

1. CD2AP and nephrin expression in developing glomeruli. The expression of both CD2AP and nephrin has been shown to be restricted to podocytes in mature mouse glomeruli. Because these molecules have been proposed to interact to stabilize the slit diaphragm, Applicants immunostained developing kidneys to determine exactly when during development CD2AP and nephrin are first expressed in podocytes. Because both the CD2AP and the nephrin antisera we used were developed in rabbit, Applicants could not double label the same section with the two antisera. Instead, Applicants stained consecutive sections individually with the two antisera and doubly labeled each with a monoclonal antibody to laminin-γ₁ to define the basic structure of the developing glomeruli. Some sections were costained with a monoclonal antibody to synaptopodin, an actin-associated protein that in the kidney is specific to podocytes. In addition, as negative controls for CD2AP staining we used age-matched CD2AP−/− kidneys, which never showed specific reactivity with the CD2AP anti-serum.

In the mouse, development of the definitive kidney begins on embryonic day 11 and continues for 2-3 weeks after birth. Because of the nature of the process, new nephrons are induced as others are maturing, and a newborn kidney exhibits all the stages of nephrogenesis.

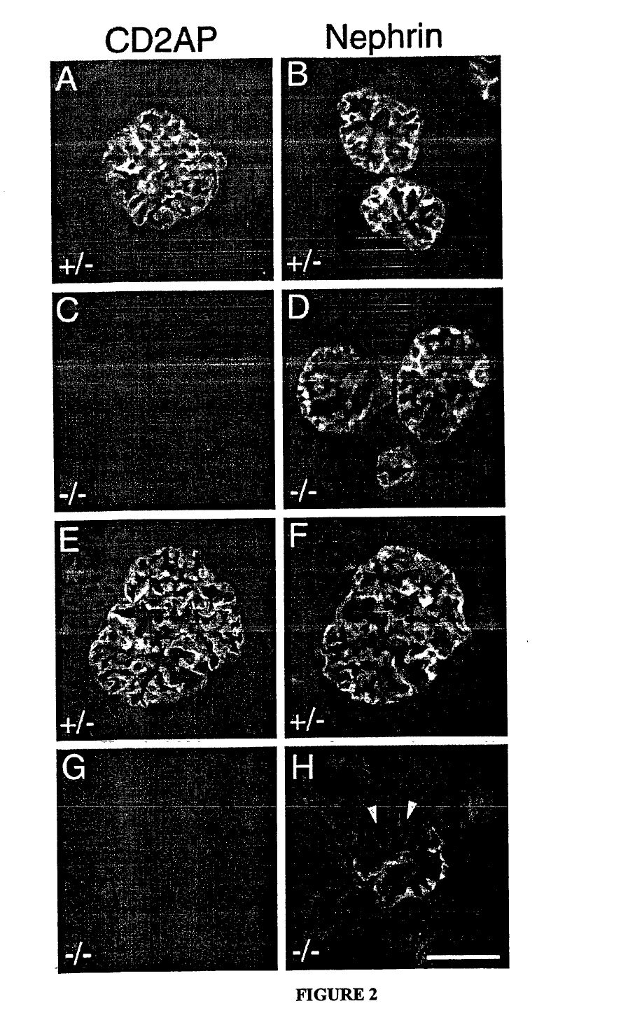

Next, Applicants immunostained 2-wk-old mouse kidneys for CD2AP and nephrin. Double labeling with the synaptopodin antibody confirmed that both proteins were restricted to podocytes in glomeruli (data not shown). Both were detected basally adjacent to the glomerular basement membrane (GBM), but there was also a diffuse labeling of the podocytes that was somewhat more extensive for nephrin (FIGs. 2, A and B). In the CD2AP −/− kidney, no staining was observed with the CD2AP antibody, whereas anti-nephrin staining was similar to that seen in the control kidney (FIG. 2, C and D). In adult glomeruli (7 weeks of age) CD2AP was less diffuse and more basally concentrated, whereas nephrin maintained a diffuse pattern restricted to podocytes (FIG. 2, E and F). In CD2AP −/− glomeruli, CD2AP was undetectable (FIG. 2G), and the same was now true for nephrin in the great majority of glomeruli. This is probably due to the fact that by 7 wk all glomeruli were severely damaged by mesangial matrix deposition, chronic nephritic syndrome, and widespread foot process effacement. Segmental nephrin reactivity was, however, observed in occasional glomeruli (FIG. 2H).

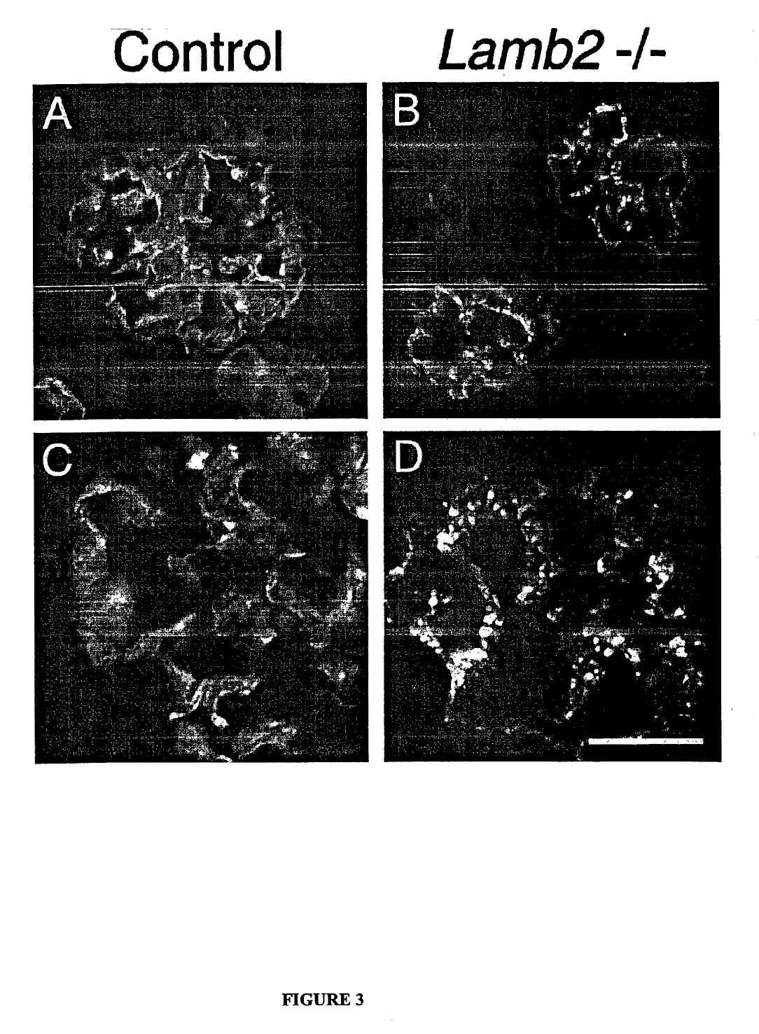

2. Localization of CD2AP in Lamb2 mutant glomeruli. Because Applicants hypothesize that CD2AP is involved in maintaining the structure of the slit diaphragm, Applicants looked at its localization in glomeruli of Lamb2 mutant mice. These mice lack the laminin-β₂ chain, a major component of the GBM and the neuromuscular synaptic basement membrane. They initially present with proteinuria at 8 days of age, exhibit extensive foot process effacement by 15 days of age, and die from renal and neuromuscular defects at 3-5 weeks of age. At 9 days of age, when there is little ultrastructural podocyte damage, no differences in CD2AP localization were noted between control and mutant mice (data not shown). However, at 21 days of age, CD2AP was found in a primarily punctate distribution in most mutant glomeruli, a pattern very different from that seen in control mice (FIG. 3). Thus, CD2AP is aberrantly localized in podocytes exhibiting or undergoing foot process effacement.

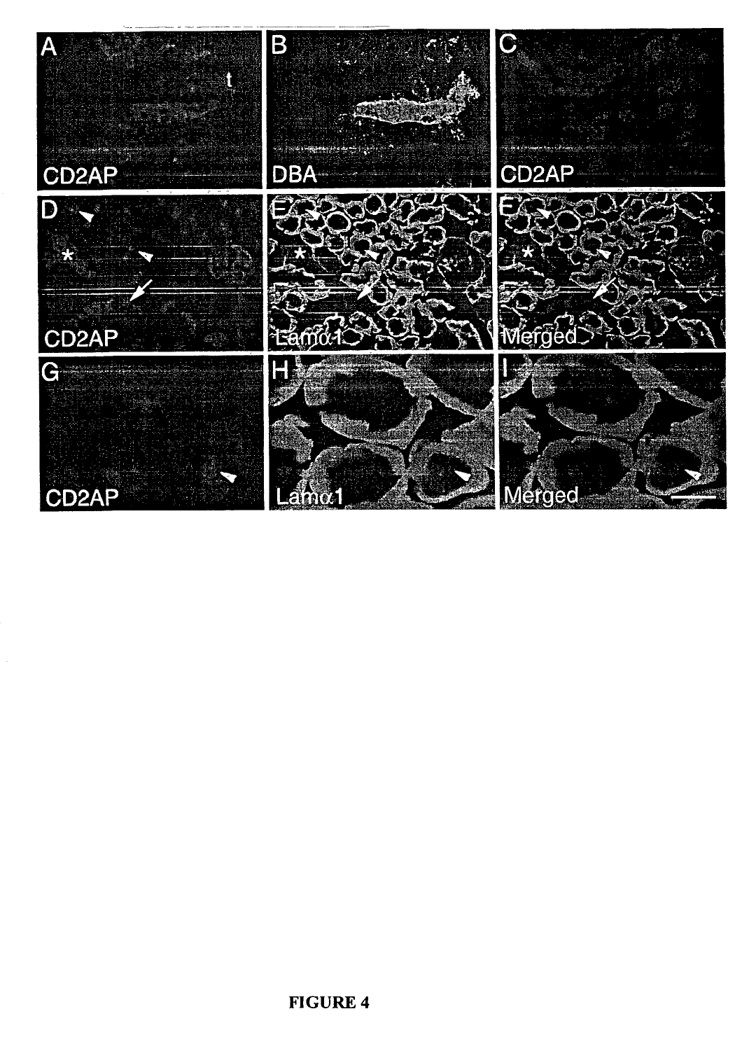

3. Extraglomerular expression of CD2AP in mouse kidney. Applicants previously reported that CD2AP was expressed in a subset of tubules. Here, Applicants have explored this issue in more detail by using markers to identify tubular segments. These included D. florus agglutinin for ureteric bud and its collecting duct derivatives, laminin-α₁ antibody, and L. tetragonolobus agglutinin for proximal tubules, and Tamm-Horsfall antiserum for thick ascending limb and segments of distal tubule (FIG. 4 and data not shown). At birth, CD2AP was detected strongly in the cortical ureteric bud epithelium, although expression was weak in the distal branching tips (FIG. 4, A and B). The medullary collecting ducts also exhibited robust CD2AP expression (FIG. 4C); this expression was consistently diffuse within these epithelial cells.

In the 2-week-old kidney, CD2AP was expressed in proximal and distal tubules where it was concentrated apically (FIG. 4, D-I). In collecting ducts, CD2AP showed a diffuse cytoplasmic localization, although there was also evidence of apical concentration in some segments (FIG. 4, D-F). The pattern at 7 wk of age did not differ significantly from that seen at 2 wk (data not shown). Thus, at maturity CD2AP is highly expressed in collecting duct epithelial cells and is concentrated apically in many but not all tubular epithelial cells of the nephron.

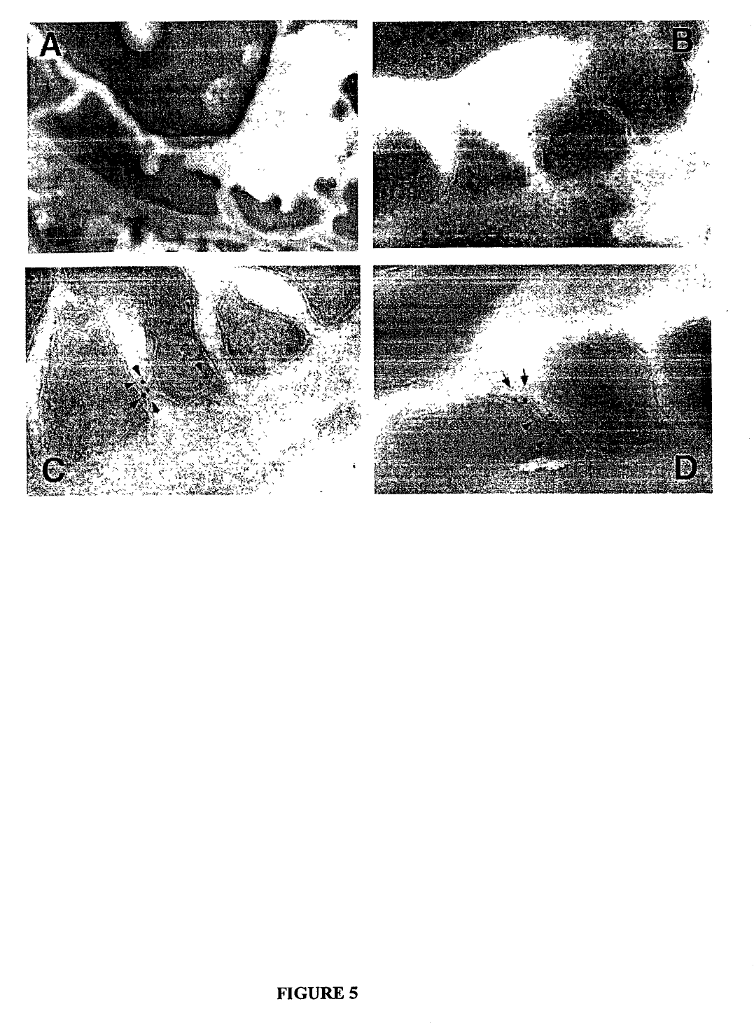

FIG. 5 is a series of photomicrographs showing the immunoelectron microscopic localization of CD2AP in a 6-week-old normal mouse was performed using affinity purified CD2AP and secondary antibody conjugated with 10 nm-gold particles. The immunogold particles (arrow heads) were found predominantly along the lateral borders of the podocyte foot processes (B-D). Many of the particles localize in a region near or at the slit diaphragm. Original magnification, ×20,000. (A) is the control.

C. Role of CD2AP in Kidney Disease

Applicants analyzed mice that are heterozygous for CD2AP, that is they have only one good copy of the CD2AP gene. First, CD2AP heterozygous mice express only about half the levels of protein as the wild type mice. In general, these mice are long lived with most animals being phenotypically normal after one year. In some of the animals, however, Applicants detected a mild proteinuria, prompting further examination of these animals.

Analysis of proteinuric heterozygous CD2AP animals demonstrated that these animals show a variety of pathologies. Most of these animals demonstrated increased cellularity in the glomeruli with increased mesangium, thickened basement membranes, and the presence of inflammatory cells. Pathologies are consistent with diseases like FSGS, membranous GN, immunotactoid GN and membranoproliferative GN. Importantly, this phenotype is distinct from the phenotype of the CD2AP knockout mouse and would not have been predicted from the knockout phenotype.

Electron microscopic analysis of the more severely infected animals demonstrated a variety of pathological changes. All of the animals demonstrate electron dense deposits that are present in the mesangium as well as in sub endothelial locations. Some of the animals demonstrated sub epithelial deposits that are diagnostic of membranous glomerulonephritis. In about one-third of animals, electron-dense deposits had a fibrillary microtubular appearance. The presence of these microtubular deposits is diagnostic for a disease entity known as immunotactoid glomerulopathy (ITG).

Immunotactoid glomerulopathy is defined as glomerular deposits that are amyloid negative, congo red negative, and with a fibrillary appearance. Studies on these deposits demonstrate that they are composed of immunoglobulin as well as the complement component C3. The etiology of ITG is completely unknown. Some pathologists postulate that ITG develops secondary to an overproduction of immunoglobulin and is a variant of membranous GN. Others believe that immunotactoid GN is a distinct clinical entity. Although not intending to be bound to any one theory, Applicants believe that ITG represents the inability of the podocyte to clear immunoglobulin rather than a process that is secondary to immunoglobulin overproduction.

The summary of Applicants' data to date on the CD2AP heterozygous mice suggests that proteinuria occurs in many but not all the animals between 12 and 24 months of age. 20-25% of the heterozygous animals are completely normal. This suggests that the disease has an incomplete penetrant pattern of inheritance. The others exhibit a range of pathologies which in human would be consistent with diagnoses such as focal segmental glomerulosclerosis, membranous glomerulonephritis, membrano-proliferative glomerulonephritis and immunotactoid glomerulophy.

As “immunotactoid” deposits are known to contain high concentrations of immunoglobulin and complement factors, Applicants postulate that CD2AP may be involved in clearing immune complexes that are trapped in the kidney. Applicants propose that environmental insults such as infectious agents increase the immune complex burden on the kidney such that the intrinsic capacity to clear such complexes is exceeded resulting in the retention of such proteins in the kidney. It is well accepted that trapped immune complexes in the kidney are responsible for the majority of kidney damage that results in glomerular damage leading to kidney failure. One novel aspect of the present invention is that genetics will play a role in determining the intrinsic capacity to clear such complexes. These data also suggest that the pathological descriptors used to classify glomerular diseases are related by a common pathway of renal injury.

Based on the phenotype of heterozygous mouse, Applicants began to explore the hypothesis that many glomerular disease such as FSGS, membranous GN, membranoproliferative GN, immunotactoid GN and lupus nephritis may be secondary to genetics in addition to external factors like autoimmunity and infectious agents. Current models suggest that glomerular disease is caused by immune complexes that are overproduced in autoimmune diseases or after infections that become trapped in the kidney and instigate an inflammatory response that cause the disease.

Based on the experiments described herein, reduced expression of CD2AP results in increased susceptibility to glomereular disease. Therefore, methods used to detect reduced CD2AP expression may be used as a diagnostic tool. For example, if a patient has a mutation in the CD2AP gene (like a mutation listed in Table 1) and expresses less CD2AP, then demonstrating decreased CD2AP expression via direct staining of the patient's kidney biopsy with an antibody will be useful to provide insight into the patient's condition. It is possible that mutations in the promoter CD2AP expression cause defects in expression. This would also be detected in such a screen. Alternatively, the proteins (transcription factors) that drive CD2AP expression might be defective, or some pathological state might inhibit CD2AP expression, all of which would result in lower levels of CD2AP expression, which could be measured using the inventive screening method. Consequently, any change in expression of CD2AP can be used to measure to assess kidney function or diagnose disease, and can be used in combination with other methods for the same.

D. Nucleic Acid Compositions Encoding CD2AP and SNPs in CD2AP Gene

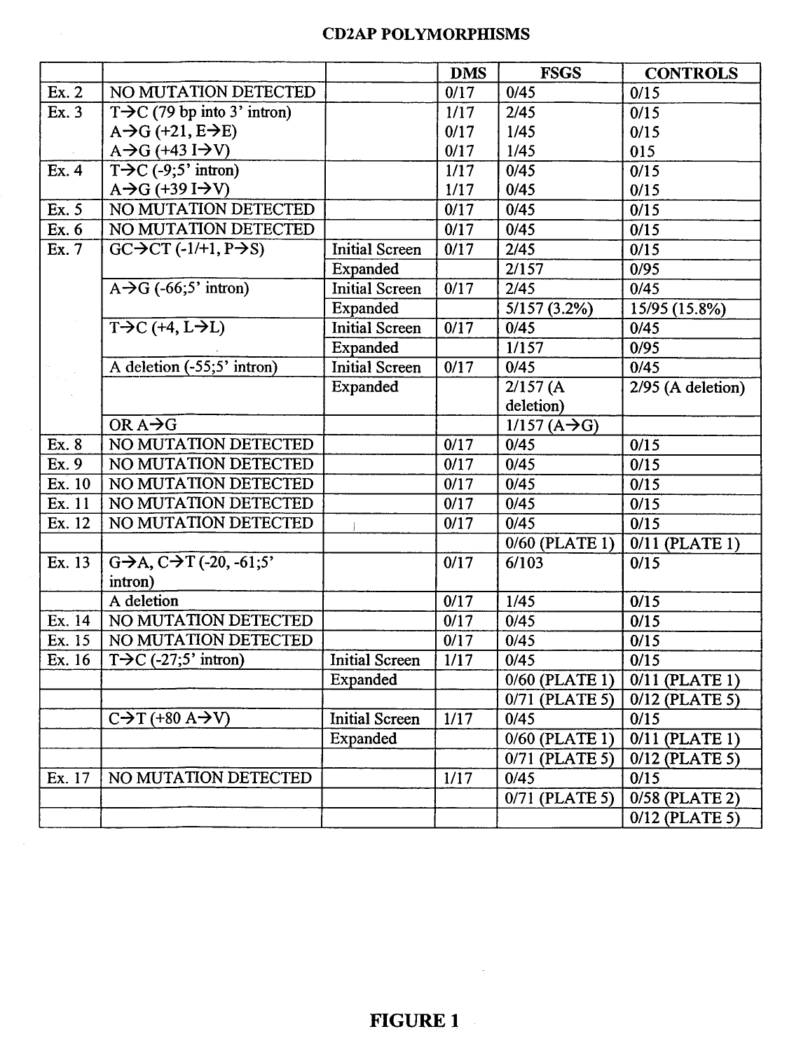

As detailed in FIG. 1 and Table 1, applicants have identified intronic, exonic, and regulatory polymorphisms in the gene for CD2AP in patients with glomerular diseases. These SNPs and gene fragments thereof are useful in the identification of predisposition to kidney disease, and for the modulation of gene activity in vivo for prophylactic and therapeutic purposes. As established herein, haplo insufficiency in humans can lead to a susceptibility to kidney disease.

The CD2AP gene has been sequenced and its regulatory regions characterized. As illustrated in FIG. 1, the gene spans approximately 143 kilobases and has 18 exons. The mature 80-kilodalton protein is encoded in exons 2-17. See Dustin et al., A Novel Adapter Protein Orchestrates Receptor Patterning and Cytoskeletal Polarity in TCell Contacts, CELL, 94:667-677 (Sep. 4, 1998). As detailed herein, the polymorphisms of the present invention have been identified and isolated and are summarized in the following Table 1.

TABLE 1
Polymorphisms in CD2AP Gene

| Coding | Genomic | Wild- | | Amino Acid | | | |
|---|---|---|---|---|---|---|---|
| Region | Location | Type | Polymorphism | Change | DMS* | FSGS+ | Controls |
| Exon 3 | 79 bp into | T | C | | 1/17 | 2/45 | 0/15 |
| | 3′ intron | | | | | | |
| Exon 3 | +21 | A | G | Glu→Glu | 0/17 | 1/45 | 0/15 |
| Exon 3 | +43 | A | G | Ile→Val | 0/17 | 1/45 | 0/15 |
| Exon 4 | −9 bp into | T | C | | 1/17 | 0/45 | 0/15 |
| | 5′ intron | | | | | | |
| Exon 4 | +39 | A | G | Ile→Val | 1/17 | 0/45 | 0/15 |
| Exon 7 | −1/+1 | GC | CT | Pro→Ser | 0/17 | 2/45 | 0/15 |
| | | | | | | and | and |
| | | | | | | 2/157 | 0/95 |
| Exon 7 | −66 6p | A | G | | 0/17 | 2/45 | 0/45 |
| | into 5′ | | | | | and | and |
| | intron | | | | | 5/157 | 15/95 |
| Exon 7 | +4 | T | C | Leu→Leu | 0/17 | 0/45 | 0/45 |
| | | | | | | and | and |
| | | | | | | 1/157 | 0/95 |
| Exon 7 | −55 bp into | A | A deletion or | | 0/17 | 0/45 | 0/45 |
| | 5′ intron | | G | | | and | and |
| | | | | | | 2/157 | 2/95 |
| | | | | | | (A | (A |
| | | | | | | deletion) | deletion) |
| | | | | | | and | |
| | | | | | | 1/157 | |
| | | | | | | (A→G) | |
| Exon 13 | −20, −61 | G | A | | 0/17 | 6/103 | 0/15 |
| | bp into 5′ | C | T | | 0/17 | 6/103 | 0/15 |
| | intron | A | A deletion | | 0/17 | 1/45 | 0/15 |
| Exon 16 | −27 bp into | T | C | | 1/17 | 0/45 | 0/15 |
| | 5′ intron | | | | | and | 0/11 |
| | | | | | | 0/60 | 0/12 |
| | | | | | | and | |
| | | | | | | 0/71 | |
| Exon 16 | +80 | C | T | Ala→Val | 1/17 | 0/45 | 0/15 |
| | | | | | | and | 0/11 |
| | | | | | | 0/60 | 012 |
| | | | | | | and | |
| | | | | | | 0/71 | |

*The number of patient samples having the particular polymorphism out of 17 samples from patients with DMS.
+This ratio shows the frequency of the particular polymorphism in samples from patients with FSGS.

To estimate the frequency of specific SNPs in the CD2AP gene, Applicants obtained DNA from over 500 subjects with glomerular disease and over 200 subjects without kidney disease. The CD2AP gene was amplified in fragments. Each fragment was then sequenced and the individual sequences were compared with the published sequence of the CD2AP gene. Deviations from the published sequence were identified as polymorphisms. Each polymorphism was then analyzed with respect to the presence or absence of kidney disease in the subject. From a preliminary analysis of approximately 139 subjects, several polymorphisms were identified that only occurred in the subjects with kidney disease (See Table 1). Thus, the presence of one or more such polymorphisms is believed to adversely change the amino acid content or expression levels of CD2AP thereby leading to kidney disease. Population-based studies to confirm that these polymorphisms are over-represented in patients with kidney disease are underway.

Frequently, the polymorphism itself is not phenotypically expressed, but is linked to sequences that result in a disease predisposition. However, in other cases, the SNP itself may affect gene expression. The use of SNPs markers for genotyping is well documented. See, e.g., Manfield et al., 24 GENOMICS 225-233 (1994); Ziegle et al., 14 GENOMICS 1026-1031 (1992).

Significant polymorphisms have been detected in exon 7. The first polymorphism (GC-CT) changes the splice acceptor site for exon 7. The nucleotide pair AG is found in 100% of splice acceptor sites. In these patients, it has been mutated to AC. Immunoblotting and sequencing of mRNA from these patients confirm that aberrant splicing occurs resulting in no detectable protein expression from this allele. These patients express about one-half the levels of CD2AP as the wild type. This is the first CD2AP mutation discovered that ablates expression of the full-length gene product. These patients therefore represent the first patients in the human population with heterozygous expression of CD2AP. Applicants have screened about 139 patients and detected this mutation three (3) times and have not detected it in over 250 control samples. Applicants believe that there exist persons who are clearly haplo insufficient yet remain disease free and undetected.

Another interesting polymorphism is also close to exon 7. This mutation (A→G) occurs in the intron preceding exon 7. Although this is in the non-coding sequence, it is in an area of the intron that would be predicted to form the lariat structure important in the splicing reaction. This mutation is found in 15/105 (14.3%) of controls while it is only found in 5/157 of patients with FSGS. This suggests that the presence of this polymorphism may be protective against the disease.

As many glomerular diseases, i.e., FSGS, are much more prevalent in the African American population, it is not surprising that Applicants found a polymorphism that is enriched in the African American population and which might increase the chances to progress to glomerular disease.

Applicants also believe that CD2AP mutations are involved in other human diseases that affect the kidney, for example systemic lupus erythemetosis (SLE). This disease is characterized by the presence of autoantibodies, usually to nuclear components like histones and DNA. This disease is also characterized by accumulation and trapping of these autoantibodies in the glomerulus and is thought to lead to glomerular nephritis and progression to kidney failure. The genetics of lupus suggest that many genes are involved in this disease. These genes are thought to influence the propensity of an individual to develop antoantibodies as well as determine their susceptibility to autoantibody mediated kidney damage. For example, many patients can be identified with antibodies to DNA and histone, but these patients do not necessarily develop the kidney disease that is a hallmark of lupus. It is well known that there is HLA linkage in lupus. Recent mapping data suggest that in humans an important disease susceptibility gene is present within the interval between 6p11 and 6p21. The CD2AP gene maps to this segment at 6p13. This is strengthened by more recent studies suggesting that the HLA linkage gene is a gene that is linked to susceptibility to kidney damage.

Another disease that may be implicated by mutations in the CD2AP gene is autosomal recessive polycycstic kidney disease (ARPKD). Applicants observed in the genomic databases that this gene is located at 6P12.3, which is the same location of CD2AP. To follow up on this observation, Applicants assessed whether CD2AP might be expressed in the cells that give rise to ARPKD, and Applicants in fact showed in Li et al. that CD2AP is expressed in distal tubule and collecting duct, which are precisely the cells that are affected in polycystic kidney disease. In addition, two reports have demonstrated protein interactions between CD2AP and the two other polycystic kidney disease proteins, PKD1 and PKD2.

The last disease implicated by Applicants' findings regarding CD2AP is diffuse mesangial sclerosis (DMS). This is a pediatric disease that closely mimics the mouse phenotype of the mouse CD2AP knockout. That is, patients with DMS are not born with proteinuria but acquire it within the first few weeks to months of life. The pathology of DMS is very similar to the pathology that Applicants have described in the CD2AP knockout mice. Applicants have screened 17 patients with DMS and have identified the polymorphisms in the CD2AP gene (see Table 1).

As shown above, the heterozygous mice for CD2AP develop a wide variety of glomerular pathologies. By EM, they demonstrate mesangial and basement membrane electron dense deposits that are enriched in complement and immunoglobulin. This suggests that heterozygous CD2AP mice have a decreased ability to clear antibodies and immune complexes from the glomerulus. Applicants postulate that decreased clearance of immune complexes leads to a variety of pathologies that include FSGS, membranoproliferative glomerulosclerosis, SLE, or immunotactoid glomerulosclerosis. CD2AP is therefore potentially very broadly involved in glomerular kidney diseases.

E. Screening for Kidney Disease by Determining Presence or Absence of Polymorphic Alleles

The invention is directed to a method of predicting the predisposition of a patient to kidney disease by genotyping the patient's DNA at the CD2AP gene cluster or CD2AP loci. The patient's genotype is compared with known CD2AP allelic variants which are known to correlate with or be associated with kidney disease.

Techniques for determining the presence of particular DNA mutations may be nucleic acid techniques based on hybridization, size, or sequence, such as restriction fragment length polymorphism (RFLP) nucleic acid sequencing, SSCP or DHPLC. These techniques may also comprise the step of amplifying the nucleic acid before analysis as is known in the art.

Kits for detecting a predisposition for kidney disease can also be employed. They can be used presymptomatically or prenatally. The diagnostic kit may comprise one or more oligonucleotides capable of hybridizing to nucleic acid from the CD2AP gene cluster. A number of assay formats are useful for genotyping using the provided oligonucleotides. The most common formats involve nucleic acid binding, such as, for example, to filters, beads, or microtiter plates and the like. Such techniques include dot blots, RNA blots, DNA blots, PCR, RFLP, and the like. The assay may also employ labeled oligonucleotides to allow ease of identification in the assays. Examples of labels which may be employed include radiolabels, enzymes, florescent compounds, streptavidin, avidin, biotin, magnetic moieties, metal binding moieties, antigen, or antibody moieties, and the like.

As discussed above, several of the polymorphisms of the present invention may be associated with kidney disease. The present invention is directed to a method of screening for kidney disease comprising determining the presence or absence of any one of the polymorphic alleles listed in Table 2, or a combination thereof, as well as other that have yet to be identified.

The following describes how these particular polymorphisms may be detected in order to screen for kidney disease. In accordance with the present invention, there are provided methods of screening for kidney disease comprising determining the presence or absence of polymorphic alleles of the CD2AP gene, wherein the presence of such an allele is indicative of kidney disease. Analysis may be of any convenient sample from a patient, e.g., cord blood sample, biopsy material, parental blood sample, etc. For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing. Samples also include biological fluids such as tracheal lavage, blood, cerebrospinal fluid, tears, saliva, lymph, dialysis fluid, and the like; organ or tissue culture derived fluids; and fluids extracted from physiological tissues. Also included are derivatives and fractions of such fluids. The cells may be dissociated, in the case of solid tissues, or tissue sections may be analyzed. Alternatively, a lysate of the cells may be prepared.

Those skilled in the art will understand that there are numerous well known methods to detect the presence or absence of a polymorphism given the sequence information provided herein. Thus, while exemplary assay methods are described herein, the invention is not so limited. For example, in one embodiment of the invention, the presence or absence of one or more polymorphic allele in a subject's nucleic acid can be detected simply by starting with any nucleated cell sample obtained from a subject from which genomic DNA, for example, can be isolated in sufficient quantities for analysis. The presence or absence of the polymorphism can be determined by sequence analysis of genomic DNA, accomplished via Maxim and Gilbert (74 PROC. NATL. ACAD. Sci. USA 560 (1977)) or Sanger (Sanger et al., 74 PROC. NAT. ACAD. SCI. 5643 (1977)) or any other conventional technique.

Amplification of nucleic acid may be achieved using conventional methods, see, e.g., Maniatis, et al., Molecular Cloning: A Laboratory Manual 187-210 (COLD SPRING HARBOUR LABORATORY, 1982). For example, mRNA from renal cells can be converted to cDNA and then enzymatically amplified to produce microgram quantities of cDNA encoding CD2AP. Amplification, however, is preferably accomplished via the polymerase chain reaction (“PCR”) method disclosed by U.S. Pat. Nos. 4,698,195 and 4,800,159, U.S. Pat. Nos. 4,683,195 and 4,683,202 or, alternatively, in a ligase chain reaction (“LCR”) (see e.g., Landegran et al., A Ligase-Mediated Gene Detection Technique, 241 (4869)) SCIENCE 1077-80 Aug. 26, 1988) and Nakazawa et al., 91 PNAS 360-364 (1994)). Alternative amplification methods include: self sustained sequence replication (Gutaelli, J. C. et al., 87 PROC NATL. ACAD. Sci. USA 1874-1878 (1990)), transcriptional amplification system (Kwoh, D. Y. et al., 86 PROC. NATL. ACAD. SCI. USA 1173-1177 (1989)), Q-Beta replicase (Lizardi, P. M. et al., 6 BIO/TECHNOLOGY 1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

The sequences complementary to the primer pairs may be separated by as many nucleotides as the PCR technique will allow. However, one of skill in the art will understand that there are practical limitations of subsequent assaying procedures, which may dictate the number of nucleotides between the sequences complementary to the primer pairs.

The amplified nucleic acid can then be assayed by any of a variety of treatment processes or methods to ascertain the genotype (and specifically the kidney disease genotype), including but not limited to: (1) allele-specific oligonucleotide probing, (2) differential restriction endonuclease digestion, (3) ligase-mediated gene detection (“LMGD”), (4) gel eletrophoresis, (5) oligonucleotide ligation assay, (6) exonuclease-resistant nucleotides, and (7) genetic bit analysis. Additional methods of analysis would also be useful in this context, such as fluorescence resonance energy transfer (“FRET”) as disclosed by Wolf et. al., 85 PROC. NATL. ACAD. SCI. USA 8790-94 (1988). SSCP and DHPLC can also be employed. Any of these or other known methods can be employed to determine the presence or absence of any one or more of the polymorphic alleles identified in Table 2 herein. The methods employed or compositions used are not intended to be limited to any one polymorphism and should be construed to encompass all polymorphisms stated herein.

1. Allele-Specific Oligonucleotide Probing (“ASO”)

One embodiment of the invention utilizes allele-specific oligonucleotide (“ASO”) probes for any of the polymorphic alleles to assay for the presence or absence of such alleles of the CD2AP gene. Accordingly, there is provided a method of screening for kidney disease, comprising assaying nucleic acid of a subject for the presence or absence of one or more polymorphic alleles of the CD2AP gene by contacting the nucleic acid with an allele-specific oligonucleotide probe(s) under conditions suitable to cause the probe to hybridize with nucleic acid encoding the polymorphic allele of the CD2AP gene, but not with nucleic acid encoding the non-polymorphic allele of the CD2AP gene, and detecting the presence or absence of hybridization.

Antisense oligonucleotides can be prepared as polynucleotides complementary to (a) nucleotide sequences comprising a DNA that encodes the polymorphic allele, or (b) nucleotide sequences comprising the polymorphic allele messenger RNA (mRNA). For both types, the length of an antisense oligonucleotide of the present invention is not critical so long as there is no promoter sequence (for DNA) or Shine-Delgarno site (for RNA) present. Type (a) antisense oligonucleotides would be synthesized de novo (DNA or RNA), or by transforming an appropriate host organism with DNA that is transcribed constitutively into RNA which binds a polymorphic allele mRNA.

According to conventional ASO procedures, oligonucleotide probes are synthesized that will hybridize, under appropriate annealing conditions, exclusively to a particular amplified nucleic acid sequence that contains a nucleotide(s) that distinguishes one allele from other alleles. The probes are discernibly labeled so that when the polymorphic allele-specific oligonucleotide robe hybridizes to the sequence encoding the polymorphic allele, it can be detected, and the specific allele is thus identified.

In a preferred embodiment of the invention, the isolated nucleic acid, which is used, e.g., as a probe or a primer, is modified such as to become more stable. Exemplary nucleic acid molecules which are modified include phosphoramidate, phosphothioate, and methylphosphonate analogs of DNA (see also U.S. Pat. Nos. 5,176,996; 5,264,564; and 5,256,775).

In one embodiment of the invention, several probes capable of hybridizing specifically to allelic variants, such as single nucleotide polymorphisms, are attached to a solid phase support, e.g., a gene chip. Oligonucleotides can be bound to a solid support by a variety of processes, including lithography. For example, a chip can hold up to about 250,000 oligonucleotides. Mutation detection analysis using these chips comprising oligonucleotides is described e.g., in Cronin et al., 7 HUMAN MUTATION 244 (1996). In one embodiment, a gene chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In another embodiment of the invention, either of the subject's amplified nucleic acid or the ASO probes can be bound onto two solid matrixes (e.g., nylon, nitrocellulose membrane, and the like) by standard techniques and then each membrane can be placed into separate hybridization reactions with an ASO probe or amplified nucleic acid, respectively. For example, if the amplified nucleic acid were bound onto a solid matrix, one hybridization reaction would utilize an oligonucleotide probe specific for the polymorphic allele under conditions optimal for hybridization of this probe to its complement. The other hybridization reaction would utilize an oligonucleotide specific to the polymorphic allele under conditions optimal for hybridization of that probe to its complement. Accordingly, the ASO probes may bear the same label, but will still be distinguishable because they are hybridized in separate chambers.

This technique permits the determination of whether the subject's nucleic acid encodes the polymorphic allele and also whether the subject is a heterozygote or a homozygote. If an ASO probe is found to bind to subject's nucleic acid on only one membrane, then the subject is homozygous for that particular allele which the ASO probe was designed to bind. If the ASO probes are found to hybridize the subject's nucleic acid on both membranes, then the subject is heterozygous. An example of this technique applied to the detection of cystic fibrosis heterozygotes is shown in Lemna, W. K., et al., 322 N. ENG. J. MED. 291-296 (1990).

The ASO probes of the present invention can be about 7 to about 35 nucleotides in length, preferably about 15 to 20 nucleotides in length, and are complementary to a nucleic acid sequence encoding at least the polymorphic nucleotide or CD2AP cDNA. Those of skill in the art will understand that other ASO probes may be designed using the sequence information provided herein. For probe design, hybridization techniques and stringency conditions, see, Ausubel, et al., (eds.) Current Protocols In Molecular Biology, Wiley Intersciences, NEW YORK, SECTIONS 6.3 AND 6.4 (1987, 1989).

The ASOs probes may be discernibly “labeled.” As used herein, the term “label” in its various grammatical forms refers to single atoms and molecules that are either directly or indirectly involved in the production of a detectable signal to indicate the presence of a complex (e.g., radioisotope, enzyme, chromogenic or fluorogenic substance, a chemiluminescent marker, or the like). Any label can be linked to or incorporated in an ASO probe. These atoms or molecules can be used alone or in conjunction with additional reagents. Such labels are themselves well known in clinical diagnostic chemistry.

One of skill in the art can readily determine such conditions for hybridization based upon the nature of the probe used, factoring into consideration, time temperature, pH, and the like.

2. Differential Restriction Enconuclease Digestion (“DRED”)

In still another embodiment of the present invention, there is provided a method of screening for kidney disease, comprising assaying nucleic acid of a subject for the presence or absence of any of the polymorphic alleles of the CD2AP gene, comprising cleaving a subject's nucleic acid with a restriction endonuclease, wherein the restriction endonuclease differentially cleaves nucleic acid encoding a polymorphic allele as compared to the wild type.

DRED analysis is accomplished in the following manner. If conditions occur including (1) a particular amplified nucleic acid contains a sequence variation that distinguishes an allele of a polymorphism and (2) this sequence variation is recognized by a restriction endonuclease, then the cleavage by the enzyme of a particular nucleic acid sequence can be used to determine the allele. In accomplishing this determination, amplified nucleic acid of a subject is digested and the resulting fragments are analyzed by size or movement through a gel. The presence or absence of nucleotide fragments, corresponding to the endonuclease cleaved fragments, determines which allele is present. A restriction endonuclease suitable for use in the practice of the present invention can be readily identified by one of skill in the art.

3. Ligase-Mediated Gene Detection (“LMGD”)

The present invention also provides methods of screening for kidney disease, comprising assaying nucleic acid of a subject for the presence or absence of any polymorphic allele, of the CD2AP gene by hybridizing the nucleic acid with a pair of oligonucleotide probes to produce a construct, wherein a first probe of the pair is labeled with a first label and a second probe of the pair is labeled with a second label, such that the first label is distinguishable from the second label, and the probes hybridize adjacent to each other. This is followed b reacting the construct with a ligase in a reaction medium, and then analyzing the reaction medium to detect the presence or absence of a ligation product comprising the first probe and the second probe.

In the course of an LMGD-type assay, a pair of oligonucleotide probes are synthesized that will hybridize adjacently to each other, for example, on a cDNA segment under appropriate annealing conditions, at the specific nucleotide that distinguishes the polymorphic alleles from the wild types. Each of the pair of specific probes is labeled in a different manner, and when it hybridizes to the allele-distinguishing cDNA segment, both probes can be ligated together by the addition of a ligase. When the ligated probes are isolated from the cDNA segment, both types of labeling can be observed together, confirming the presence of the polymorphic allele-specific nucleotide sequence. Examples of such LMGD-type assays, which one skilled in the art may easily perform, are disclosed in Rotter et al., U.S. Pat. No. 6,008,335.

4. Gel Electrophoresis

In other embodiments, alterations in electrophoretic mobility will be used to identify mutations or the identify of the allelic variant of a polymorphic region in CD2AP genes. For example, single strand conformation polymorphism (SSCP) may be used to detect differences in electrophoretic mobility between mutant and wild type nucleic acids (Orita et al., 86 PROC. NATL. ACAD. SCI. USA 2766 (1989); see also Cotton, 285 MUTAT. RES. 125-144 (1993); and Hayashi, 9 GENET. ANAL. TECH. APPL. 73-79 (1992)). Single-stranded DNA fragments of sample and control CD2AP nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in eletrophoretic mobility (Keen et al., TRENDS GENET. 5 (1991)).

In yet another embodiment, the movement of mutant or wild-type fragments in polyacrylamide gels containing a gradient of denaturant is assayed using denaturing gradient gel electrophoresis (“DGGE”) (Myers et al., 313 NATURE 495 (1985)). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner, 265 BIOPHYS. CHEM. 12753 (1987)).

5. Oligonucleotide Ligation Assay (“OLA”)

In another embodiment, identification of the allelic variant is carried out using an oligonucleotide ligation assay (“OLA”), as described, e.g., in U.S. Pat. No. 4,998,617 and in Landegren et al., 241 SCIENCE 1077-1080 (1988). The OLA protocol uses two oligonucleotides, which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al., have described a nucleic acid detection assay that combines attributes of PCR and OLA (Nickerson, D. A. et al., 87 PROC. NATL. ACAD. Sci. U.S.A. 8923-8927 (1990). In this method, PCR is used to achieve the exponential amplification of target DNA, which is then detected using OLA. Several techniques based on this OLA method have been developed and can be used to detect specific allelic variants of a polymorphic region of a CD2AP gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al., 24 Nucleic Acids Res. 3728 (1996), OLA combined with PCR permits typing of two alleles in a single microtiter well. By marking each of the allele-specific primers with a unique hapten, i e., digoxigenin and fluorescein, each OLA reaction can be detected by using hapten specific antibodies that are labeled with different enzyme reporters, alkaline phosphatase, or horseradish peroxidase. This system permits the detection of the two alleles using a high throughput format that leads to the production of two different colors.

6. Exonuclease-Resistant Nucleotides

In one embodiment, the single base polymorphism can be detected by using a specialized exonuclease-resistant nucleotide, as disclosed, e.g., in Mundy, et al. U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identify of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amount of extraneous sequence data.

In another embodiment of the invention, a solution-based method is used for determining the identity of the nucleotide of a polymorphic site. Cohen, D. et al. (French Patent, 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

7. Genetic Bit Analysis (GBA™)

An alternative method, known as GBA™ is described by Goelet, P. et al. (PCT Appln. No. 92/15712). The method of Goelet, P. et al., uses mixtures of labeled terminators and primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al., (French Patent No. 2,650,840; PCT Appln. No. WO91/02087) the method of Goelet, P. et al., is preferably a heterogeneous phase assay, in which the primer or the target molecule is immobilized to a solid phase.

F. Susceptibility to Kidney Disease

Applicants believe that environmental insults or disease states can ablate CD2AP expression completely in haplo insufficient individuals by damaging their one good CD2AP gene. Thus, it is imperative for such individuals to know of their haplo insufficiency and avoid such insults and diseases, if possible.

Those skilled in the art will appreciate that any of the foregoing inventive methods may be used not only to diagnose kidney disease, but also to predict a subject's susceptibility to kidney disease. These methods for determining susceptibility to kidney disease are particularly useful in combination with a subject's family history of kidney disease. To alleviate the concern of the parent, and to take any preventative measures which might prevent onset, one of the many inventive methods provided herein can be used to determine whether the patient is a carrier of one or more the polymorphic alleles identified herein.

Gene chips can also be employed for screening using the inventive polymorphisms.

Similarly, the screening methods provided herein are preferably used in combination with existing methods for diagnosing kidney disease (e.g., radiological and biochemical) to maximize a confidence in the ultimate diagnosis regarding kidney disease.

G. Kits

Kits for use in screening for kidney disease and other kidney diseases and screening for susceptibility to kidney disease are also provided by the present invention. Such kits can include all or some of the reagents, primers, probes, antibodies, and antisense oligonucleotides described herein for determining the presence of absence of nucleic acid encoding one or more polymorphic allele, or for treatment of kidney disease. Kits of the present invention may contain, for example, restriction endonuclease; one or more labeled oligonucleotide probes that distinguish nucleic acid encoding the relevant nucleotides of CD2AP cDNA; ligase; polymorphic allele-specific oligonucleotide probe; primer for amplification of nucleic acid encoding the relevant nucleotide of CD2AP cDNA; means for amplifying a subject's nucleic acid encoding the cDNA; neutrophil, alkaline phosphatase coupled goat anti-human gamma chain specific antibody; fluorescein-labeled goat anti-human gamma chain specific antibody; anti-human gamma chain specific antibody; antisense oligonucleotides; antibody specific for, or which binds the polymorphic allele; or combinations of any of the above.

These suggested kit components may be packaged in a manner customary for use by those of skill in the art. For example, these suggested kit components may be immobilized on a solid matrix or provided in solution or as a liquid dispersion or the like.

H. Methods of Treatment

The present invention provides for both prophylactic and therapeutic methods of treating a subject having or at risk of having kidney disease. Subjects at risk for such a disease can be identified by a diagnostic or prognostic assay as described herein. Administration of a prophylactic agent can occur prior to the manifestation of symptoms characteristic of the CD2AP disruption, such that development of kidney disease is prevented or, alternatively, ameliorated in its progression. In general, the prophylactic or therapeutic methods comprise administering to the subject an effective amount of a compound, which is capable of augmenting a wild-type CD2AP activity or antagonizing a mutant defective CD2AP activity.

Gene replacement therapies using DNA transfection or viral vectors would be expected to ameliorate the effects of CD2AP haploinsufficiency. Cells can be transfected using any appropriate means, including viral vectors, chemical transfectants, or physico-mechanical methods such as electroporation and direct diffusion of DNA. See, for example, Wolff, Jon Aal, et, Direct Gene Transfer Into Mouse Muscle In Vivo, SCIENCE, 247, 1465-1468, 1990; and Wolff, Jon A, Human Dystrophin Expression In Mdx Mice After Intramuscular Injection Of DNA Constructs, NATURE, 352, 815-818, 1991. Plasmid DNA, which can function episomally, has been used with liposome encapsulation, CaPO₄ precipitation and electroporation as an alternative to viral transfections. Clinical trials with liposome encapsulated DNA in treating melanoma is reported by Nabel, J. G., et al., Direct Gene Transfer With DNA-Liposome Complexes In Melanoma: Expression, Biological Activity And Lack Of Toxicity In Humans, PROC. NAT. ACAD. Sci. U.S.A., 90 (1993) 11307-11311; Feigner, Philip L, Lipofectamine Reagant: A New, Higlier Efficiency Polycationic Liposome Transfection Reagant, Focus/GIBCO, 15, 73-78, 1993; Partridge, Terence A, Muscle Transfection Made Easy, NATURE, 352, 757-758, 1991; Wilson, James M, Vehicles For Gene Therapy, NATURE, 365, 691-692, 1993; Wivel, et al., Germ-Line Gene Modification And Disease Prevention: Some Medical And Ethical Perspectives, SCIENCE, 262, 533-538, 1993; and Woo, Savio L Cal, et, In Vivo Gene Therapy Of Hemophilia B: Sustained Partial Correction In Factor IX-Deficienit Dogs, SCIENCE, 262, 117-119, 1993.

As used herein, vectors are agents that transport the gene into the cell without degradation and include a promoter yielding expression of the gene in the cells into which it is delivered. Promoters can be general promoters, yielding expression in a variety of mammalian cells, or cell specific, or even nuclear versus cytoplasmic specific. These are known to those skilled in the art and can be constructed using standard molecular biology protocols. Vectors have been divided into two classes:

a) Biological agents derived from viral, bacterial or other sources.

b) Chemical/physical methods that increase the potential for gene uptake, directly introduce the gene into the nucleus or target the gene to a cell receptor.

Biological Vectors

Viral vectors have higher transaction (ability to introduce genes) abilities than do most chemical or physical methods to introduce genes into cells.

Retroviral vectors are the vectors most commonly used in clinical trials, since they carry a larger genetic payload than other viral vectors. However, they are not useful in non-proliferating cells. Adenovirus-vectors are relatively stable and easy to work with, have high titers, and can be delivered in aerosol formulation. Pox viral vectors are large and have several sites for inserting genes, they are thermostable and can be stored at room temperature. Lentivirus vectors can also be employed.

Plasmids are not integrated into the genome and the vast majority of them are present only from a few weeks to several months, so they are typically very safe. However, they have lower expression levels than retroviruses.

Chemical/Physical Vectors

Other methods to directly introduce genes into cells or exploit receptors on the surface of cells include the use of liposomes and lipids, ligands for specific cell surface receptors, cell receptors, and calcium phosphate and other chemical mediators, microinjections directly to single cells, electroporation and homologous recombination. Liposomes are commercially available from Gibco BRL, for example, as LIPOFECTION® and LIPOFECTACE®, which are formed of cationic lipids such as N-[1-(2,3dioleyloxy)-propyl]-n,n,n-trimethylammonium chloride (DOTMA) and dimethyl dioctadecylammonium bromide (DDAB). Numerous methods are also published for making liposomes, known to those skilled in the art.

Delivery

The inventive vector can be administered, for example, via cannulation of the renal artery.

The contents of all cited references throughout this application are hereby expressly incorporated by reference. The practice of the present invention will employ, unless otherwise indicated, conventional techniques of pharmacology and pharmaceutics, which are within the skill of the art. Unless defined otherwise, all technical and scientific terms used herein have the same meaning as is commonly understood by one of skill in the art to which this invention belongs.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

We claim:

1. A method of predicting a mammalian subject's susceptibility to kidney disease, comprising:
a) providing
i) a sample from the subject, wherein the sample comprises nucleic acid, the nucleic acid comprising a CD2AP gene, and
ii) a treatment process;
b) treating the sample with the treatment process under conditions such that a genotype for kidney disease is detected if present, wherein the genotype comprises a genotype homozygous for at least one allele of a plurality of polymorphic sites of the CD2AP gene listed in Table 1; and
c) detecting the disease genotype if present, wherein the presence of the genotype is indicative of the subject's susceptibility to the disease.

2. The method of claim 1 wherein the treatment process comprises at least one of: allele-specific oligonucleotide probing, differential restriction endonuclease digestion, SSCP, DHPLC, ligase-mediated gene detection, gel electrophoresis, oligonucleotide ligation assay, exonuclease-resistant nucleotides, genetic bit analysis and fluorescence resonance energy transfer.

3. The method of claim 1, wherein said sample is blood.

4. An isolated and purified nucleic acid of at least one of a plurality of polymorphisms listed in Table 1.

5. A method for screening kidney disease in a mammalian subject, comprising:
a) obtaining a sample from the subject;
b) preparing the sample for analysis by isolating at least one of DNA, RNA, or protein from the sample; and
c) determining the presence or absence of at least one CD2AP polymorphism associated with the disease within the sample by analyzing the isolated DNA, RNA, or protein using probes specific for the polymorphism.

6. A method for determining the expression of CD2AP, comprising:
a) Obtaining a tissue sample from a patient;
b) treating the sample with an antibody specific for CD2AP;
c) measuring the amount of CD2AP bound to the antibody; and
d) Analyzing the pattern of CD2AP expression in the glomerulus to make a diagnostic decision.
